(19) United States
(12) Patent Application Publication
(10) Pub. No.: US 2012/0245555 A1
(43) Pub. Date: Sep. 27, 2012

(54) Blood tube section for extracorporeally carrying blood, extracorporeal blood circuit, treatment apparatus as well as methods

(75) Inventor: Reiner Spickermann, Wasserlosen-Burghausen (DE)

(73) Assignee: FRESENIUS MEDICAL CARE DEUTSCHLAND GMBH, Bad Homburg (DE)

(21) Appl. No.: 13/428,117

(22) Filed: Mar. 23, 2012

Related U.S. Application Data
(60) Provisional application No. 61/466,951, filed on Mar. 24, 2011.

(30) Foreign Application Priority Data
Mar. 24, 2011 (DE) 102011 015 075.7

(51) Int. Cl. A61M 1/14; A61M 5/00; A61M 39/28; A61M 39/00

(52) U.S. Cl. 604/500; 604/264; 604/272; 604/250

(57) ABSTRACT

The present invention relates to a blood tube section for extracorporeally carrying blood during a blood treatment of a patient, wherein the blood tube section comprises at least one device for conveying blood, a drug solution or a liquid mixture within the blood tube section by deforming the tube diameter and sliding along an area of the blood tube section. The present invention further relates to an extracorporeal blood circuit, a treatment apparatus as well as methods for conveying blood, a drug solution or a liquid mixture and for supplying and/or administering and/or transporting an active substance.

### CROSS REFERENCE TO RELATED APPLICATIONS

This application claims the benefit of U.S. Provisional Patent Application No. 61/466,951 filed on Mar. 24, 2011 and German Patent Application No. 10 2011 015 075.7, filed Mar. 24, 2011, both of which are hereby incorporated by reference.

### FIELD OF INVENTION

The present invention relates to blood tube sections for extracorporeally carrying blood during blood treatment of patients. It further relates to an extracorporeal blood circuits, treatment apparatuses, and various methods for conveying blood and administering active substances.

### BACKGROUND OF INVENTION

Frequently, after ending a blood treatment that was performed extracorporeally, blood remains in the extracorporeal blood circuit used. This is disadvantageous for various reasons. On the one hand, blood remaining in the extracorporeal blood circuit and being disposed of together with the extracorporeal blood circuit at the end of the treatment constitutes a blood loss of the patient which adds up over subsequent blood treatments. On the other hand, any liquid remaining in the extracorporeal blood circuit after the blood treatment increases the risk of infection for third parties. Thus, the extracorporeal blood circuit will not be disposed of like ordinary household waste. It will rather be disposed of separately from household waste, as is standard also with other infectious material in the medical environment. This kind of disposal is cost-intensive and is regularly billed per kilogram waste by the waste disposal center. As a consequence, liquid remaining in the extracorporeal blood circuit at the end of the blood treatment increases the blood circuit's weight and thus also its disposal costs.

### SUMMARY OF THE INVENTION

One object of the present invention is to propose a device for reducing the amount of liquid remaining in an extracorporeal blood circuit at the end of the treatment and/or to propose a further device by which a drug may be administered to the patient.

The blood tube section according to the present invention comprises at least one tube (alternatively referred to as a piece of tube) having a lumen which is open in the longitudinal direction as well as a device for conveying a liquid such as, e.g., blood, drug solutions or liquid mixtures within the tube. The device is designed such that conveying is performed by deforming or changing the shape of the tube diameter and by moving, sliding, shifting or driving the device along an area of the tube.

A device for conveying a fluid like blood, drug solutions or liquid mixtures within an extracorporeal tube, or for delivering it from the extracorporeal blood tube or extracorporeal blood circuit by the blood tube section according to the present invention is provided.

The extracorporeal blood circuit according to the present invention is connected in fluid connection with at least one blood tube section according to the present invention.

The treatment apparatus according to the present invention is connected, in particular in fluid connection, with at least one blood tube section according to the present invention and/or at least one extracorporeal blood circuit.

The method according to the present invention for conveying a fluid—like, e.g., blood, a drug solution or a liquid mixture—within a blood tube or a blood tube section and/or for delivering blood from a blood tube or a blood tube section encompasses using a blood tube section according to the present invention. In doing so, its conveying device is directed along the blood tube section or the blood tube, e.g., manually or mechanically.

The method according to the present invention for supplying and/or administering and/or transporting an active substance within a blood tube encompasses using a blood tube section according to the present invention, administering the active substance, directly or indirectly, into the lumen of the tube, and conveying the active substance within the lumen by shifting or moving the conveying device along the tube or a section of the tube.

The method according to the present invention for returning blood from a blood tube, a blood tube system or an extracorporeal blood circuit encompasses using a blood tube section according to the present invention which is in fluid connection with the blood vessel of a patient through a needle. The method further encompasses—in particular manually or mechanically—directing the device for conveying blood present in the blood tube section, at the end of the blood treatment of the patient, along the tube or a section hereof. The latter takes place for the purpose or aim of partially or completely conveying or returning blood present in the blood tube section into the blood vessel of the patient.

Developments of the present invention are each the subject of dependent claims and embodiments according to the present invention.

Embodiments according to the present invention may comprise one or more of the features named hereafter.

In all of the following embodiments, the use of the expression may be or may have and so on, is to be understood synonymously with preferably is or preferably has, respectively, and so on, and is intended to illustrate certain embodiments according to the present invention.

In certain embodiments according to the present invention, a blood tube section is a section or segment or part of a larger, longer or superior blood tube system, blood tube set or blood circuit, each provided for extracorporeally carrying blood, or each provided for being connected hereto. A blood tube system may comprise one or more blood tube sections. A blood tube system may—in the sense of the present invention—be referred to as a blood tube set.

Carrying blood, as used herein in certain embodiments, refers to a directed moving or conducting of blood as is known in connection with tubes, lines—such as, e.g., arterial or venous patient lines—and so on.

In some embodiments according to the present invention, a drug solution is a drug or a pharmacological and/or pharmaceutical active substance (drug and active substance are partially used synonymously hereafter). A drug solution may be a combination of drugs and/or active substances. Drugs or active substances are hereby present in a dissolved or suspended state. For instance, they are introduced in a physiological saline solution.

In some embodiments according to the present invention, the blood tube section consists of one or more conveying devices in addition to a piece of tube.

In other embodiments, the blood tube section additionally comprises conclusively or not conclusively one or more ports for indirectly or directly establishing a fluid connection between the blood tube section or the tube on the one hand and a needle for puncturing a blood vessel of the patient on the other hand.

The ports may—each singly and independently of other ports—be integral with the tube or may have been connected to the tube after its production.

In certain embodiments according to the present invention, the blood tube section additionally comprises conclusively or not conclusively one or more ports for indirectly or directly establishing a fluid connection between the blood tube section or the tube on the one hand and a blood tube system on the other hand.

In certain embodiments according to the present invention, the blood tube section according to the present invention comprises one or more ports which are designed for a standardized or suited connection for tube systems in the medical field. Preferably, such port is embodied as a Luer-Lock connector.

In some embodiments according to the present invention, the blood tube section additionally comprises conclusively or not conclusively one or more needles for indirectly or directly establishing a fluid connection between the blood tube section or the tube on the one hand and the blood vessel of the patient on the other hand.

The one or more needles are designed in certain embodiments according to the present invention in order to be connected with the blood tube section according to the present invention by use of a standardized connecting system, preferably a Luer-Lock system.

In some embodiments according to the present invention, the blood circuit further comprises conclusively or not conclusively one or more puncturing aids for facilitating puncturing the blood vessel of the patient and/or for fixing the blood tube section to a skin portion or a different body portion (such as the wrist) of the patient.

Such puncturing aid may comprise stiff or flexible hold-up aids that are connected with the needle. With the help of these hold-up aids, the needle may, e.g., be guided with the hand such that a puncture in a blood vessel of the patient may be performed easily and safely for the patient and/or the doctor.

Some puncturing aids may be used after puncturing for fixing the needle system onto a skin portion of the patient for a longer length of stay of, e.g., several hours or days.

Devices known under the name Butterfly are among the better-known puncturing aids—as used in certain embodiments according to the present invention. However, puncturing aids are not limited to a design of this sort.

In certain embodiments according to the present invention, the tube of the blood tube section is made of or comprises a flexible or elastic and/or transparent or opaque (for the purpose of the possibility of a visual inspection from outside) material.

In some embodiments according to the present invention, a deformation of the tube diameter takes place by compressing or squeezing the tube using the conveying device. Compressing or squeezing the tube may take place manually or mechanically.

One example for a suchlike deformation of a tube may be observed, e.g., in the area of a roller pump when the roller pump conveys blood or drug solutions through extracorporeal tube systems.

In some embodiments according to the present invention, the tube—e.g., in its compressibility and/or its diameter (inner and/or outer diameter)—and the device for conveying are coordinated.

This coordination may for instance be such that the conveying device in its closed or locked-in state influences the tube such that the conveying device remains movable and influences the tube contents with defined or predetermined or constructively limited force. This way, it may be ensured that, e.g., a complete clamping does not occur, and/or that blood cells are damaged not at all or only to an acceptable amount.

In certain embodiments according to the present invention, blood, drug solutions or liquid mixtures are conveyed by deforming the tube diameter and active sliding, shifting, moving or the like of the conveying device along an area of the blood tube section. Hereby, a remaining cross-section of the lumen of the tube may stay open; in other embodiments according to the present invention, the lumen is closed completely.

In certain embodiments according to the present invention, the device according to the present invention for conveying blood is designed as or comprises a tubing clamp.

The term tubing clamp, as used herein in certain embodiments, describes an apparatus through which the lumen or the internal cross-sectional area of the tube may be reduced or deformed completely or almost completely. This may be achieved by compressing or squeezing the tube from outside by use of the conveying device such that no or almost no fluid flows within the tube.

In certain embodiments according to the present invention, the conveying device may close the tube lumen (or the lumen of the tube) or the tube in at least a cross-section hereof with intended utilization of the blood tube section partially, in some embodiments according to the present invention completely. In corresponding embodiments according to the present invention of the method, the conveying device is used exactly the one way or the other way.

In some embodiments according to the present invention, the conveying device may close the lumen of the tube steplessly, i.e., continuously variable, however, in certain embodiments according to the present invention it may close the lumen of the tube in several steps or by use of only one step (i.e., the device is either open or not open). Closing in one step may for instance be achieved by use of a snap lock or latch lock which comprises only one closed position in which the snap lock latches. Closing in several steps may for instance be achieved by use of a snap lock or a latch lock which comprises more than only one closed position in which the snap lock may latch.

In certain embodiments according to the present invention, the device according to the present invention for conveying comprises one or two rollers—each independently of the design of other rollers—that are rotatably or not rotatably mounted, which during delivering the fluid, e.g., the blood, narrow the lumen of the corresponding section of the tube singly, individually or jointly.

In some embodiments according to the present invention, each roller may independently of the design of further rollers be embodied as rotary cylinder having two roller pins or studs on the face side. The roller pins may be provided to be the axis of a bearing.

The bearing may, e.g., be a slide bearing, wherein the design and choice of material of such slide bearings is known to the person skilled in the art from, e.g., the construction of cylindrical roller bearings.

In case there are at least two rollers—as proposed in some embodiments according to the present invention—arranged in or on the conveying device, e.g., embodied as tubing clamp, these two or more rollers may, in a conveying state, deform or squeeze the tube between each other, based on or originating from two opposing sides. Hereby, the lumen of the tube or the tube lumen may be closed completely or substantially completely in an, e.g., one-step closing.

In relation to the longitudinal extension, the rollers may be arranged in the conveying device such that they are opposite to each other, e.g., within a plane which is perpendicular to the axial direction and/or for instance point-symmetrically to this plane. At least two rollers may, however, also be arranged to be staggered at two different spots in the longitudinal direction of the tube. The stagger or displacement or offset may, e.g., be between 1 and 20 mm, preferably between 5 and 15 mm, particularly preferably between 8 and 12 mm.

Each of the rollers used here may—independently of the design of further rollers—be designed, i.a., as barrel roller, ball bearing, rolling bearing or the like, or may be replaced herewith.

In certain embodiments according to the present invention, the one roller or the more rollers are fixed to or on the conveying device to be detachable again.

Axes of the rollers may be fixed with their front side areas each in one plastic snap lock of the device. The latter may be embodied as slide bearings.

In certain embodiments according to the present invention, at least one roller protrudes from the conveying device in one section hereof. This may advantageously contribute to enabling a one-hand operation or one-hand movement of the conveying device in a comfortable manner by actuating the protruding section of the roller in a rolling way—e.g., through use of the thumb.

In some embodiments according to the present invention, the conveying device is, inter alia, also provided for clamping, i.e., completely closing the tube lumen. The device may thus advantageously fulfill different tasks.

In certain embodiments according to the present invention the conveying device is not, in particular not slidably or not firmly, attached to a treatment apparatus, e.g., a dialysis machine, and is no component or section thereof. In fact, in these embodiments the conveying device is comprised by the blood tube section.

In certain embodiments according to the present invention, the blood tube section comprises an addition point for adding liquid into the lumen of the blood tube section.

In some embodiments according to the present invention, the addition point is provided and designed for adding one or more liquid substances such as solutions, suspensions and the like.

The addition point may be, e.g., a connector, a stub or branch line, a penetration membrane, a port for the blood tube set or blood circuit, e.g., a Luer-Lock or a Luer connector, a membrane for a pressure-driven, concentration-driven, thermally- or electrically-driven mass transfer or process through the membrane, and so on.

The addition point may be provided for supplying the blood tube section with one or more drugs or active substances.

In some embodiments according to the present invention, a drug or a liquid mixture of blood with one or more drugs is conveyed by the conveying device. Hereby, a drug may be present as a drug solution, e.g., dissolved in physiological saline solutions or dispersed as suspension.

Conveying in the sense of the present invention occurs in some embodiments according to the present invention within the blood tube section in a direction towards the vascular system of the patient.

In some of these embodiments, this direction towards the vascular system is referred to as second direction or second direction of flow. This second direction is contrary to a first direction (a first direction of flow) in which blood, a drug or a different fluid flows through or mainly flows through the blood tube section or the whole extracorporeal blood circuit during the treatment of the patient. For instance, in certain embodiments according to the present invention, it is intended to move blood, a drug or a different fluid within the blood tube section in the direction towards the arterial needle by use of the conveying device or by applying the method according to the present invention, wherein blood, a drug or a different fluid flows in the opposite direction through the blood tube section which, e.g., is situated in the direction of flow (during the blood treatment) between the arterial patient needle on the one hand and the blood pump or blood treatment apparatus, e.g., dialysis filter, on the other hand, during the blood treatment itself.

In certain embodiments according to the present invention, the blood, the drug solution or the liquid mixture is delivered out of a blood tube or the blood tube section according to the present invention completely or partially.

In this context, delivering means complete or partial draining of the tube. In some embodiments according to the present invention, the vascular system of a patient is in fluid connection with the tube during delivering. In other embodiments according to the present invention, this is not the case. In the latter cases, the delivered blood is not supplied to the vascular system of the patient by the method according to the present invention, but otherwise collected and optionally further used or utilized, or not.

The treatment apparatus according to the present invention is in some embodiments according to the present invention a blood treatment apparatus. It may particularly be provided and embodied for performing a hemodialysis, hemofiltration or hemodiafiltration of a patient.

Certain embodiments according to the present invention comprise one or more advantages named hereafter and/or above.

The present invention provides a blood tube section for extracorporeally carrying blood, but also drug solutions or the like, during or at the end of a blood treatment of a patient. The blood tube section comprises a device for conveying and transporting blood or drug solutions within the blood tube section. Through the use of this device, the tube is at first deformed, wherein deforming may encompass squeezing. In this state or due to this state, its tube contents are moved along the blood tube section. This way, e.g., a drug may also be conveyed and administered after a shutdown of a blood pump.

Through the use of the blood tube section according to the present invention, at least a part of the blood remaining in extracorporeal blood circuits at the end of dialysis treatments may advantageously be returned to the treated patient. Due to the manual operability and the possibility of visual control of the tube contents' quality, precise returning of blood into the body is possible safely and with high yield and/or accuracy.

In using the blood tube section according to the present invention, it may advantageously be possible to avoid even that small blood volumes remain in the tube system.

Further, because this can be carried out manually under visual control, it is advantageously possible to avoid infusing additional liquid such as, e.g., saline solution into the vascular system when returning blood into the vascular system. This is not always avoidable with methods of the state of the art, however, it is of relevance especially for the dialysis patient. Additionally, there is no risk according to the present invention of, e.g., air infusion, blood clot infusion and the like.

A further advantage in using the blood tube section according to the present invention is that in using it, drugs may be administered without an additional puncture of the vascular system of the patient. The blood tube section is suited for being supplied into its interior—the conveying device serves to drive the administered substance.

The utilization of saline solution, syringe material and the like is advantageously not necessary for returning blood from the blood tube section into the vascular system of the patient. Thus, costs associated herewith and also a hygienic risk may be avoided.

Providing rollers may advantageously reduce friction that might occur during moving the conveying device along the tube. This may in individual cases contribute to the fact that the conveying device may be moved along the tube with only little effort, and even more importantly even with only one hand.

Providing replaceable or detachable rollers may allow that the conveying device may be adapted to the thickness of the tube or other conditions by using rollers that are particularly suited for the individual case. This may advantageously reduce the effort for producing the conveying device or make its base body universally usable due to use of differently designed rollers.

### BRIEF DESCRIPTION OF THE DRAWINGS

Hereinafter, the present invention is exemplarily explained by the figures of the appended drawings in which identical reference numerals refer to same or identical components. In the partly simplified figures, it applies:

FIG. 1 shows a blood tube section according to the present invention;

FIG. 2 shows a device for conveying, e.g., blood of a blood tube section according to the present invention; and

FIG. 3 shows—schematically simplified—a treatment apparatus according to the present invention on which two blood tube sections according to the present invention are arranged.

### DETAILED DESCRIPTION OF THE DRAWINGS

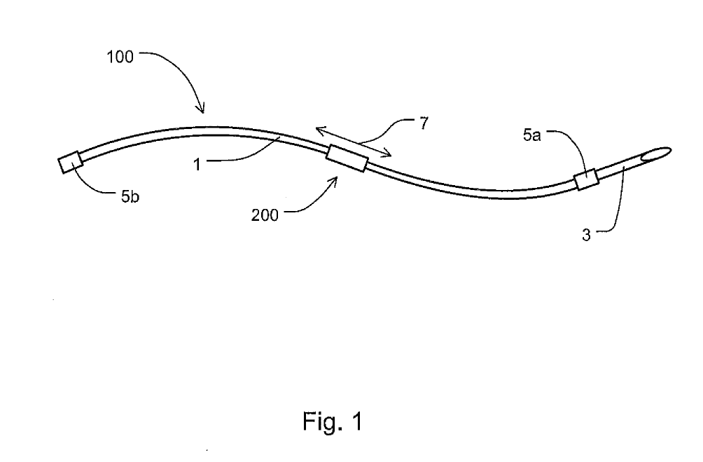

FIG. 1 shows a blood tube section 100 according to the present invention for extracorporeally carrying blood, drug solutions or liquid mixtures.

The blood tube section 100 comprises a tube 1, a needle 3, two ports or connectors 5a, 5b and a device 200 for conveying tube contents along the tube 1.

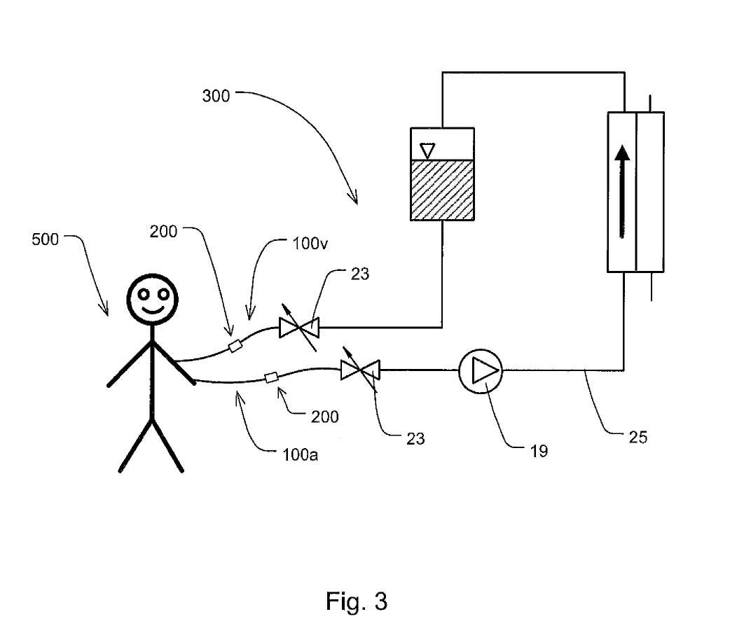

The needle 3 is connected with the blood tube section 100 by a connector 5a at one end. The needle 3 may, e.g., be used for puncturing a vessel of a patient, in order to infuse blood, a drug solution or a liquid mixture from the tube 1 into a vessel of a patient 500 (see FIG. 3) or in order to withdraw blood from the vessel.

At the other end of the blood tube section 100, a further connector 5b is fixed or provided in one piece,through which the blood tube section 100 may be connected to a tube system such as a dialysis tubing set or the like, a storage container or a different apparatus.

The connector 5b may serve as addition point for drugs or fluids in general. These may be introduced into the lumen of the tube 1 via the connector 5b through, e.g., a cannula or a squeezable drug container.

Through use of the conveying device 200, which is movable in one or both directions indicated by the arrow 7, blood, a drug solution or a liquid mixture may be transported or conveyed into the tube 1, within the tube 1 and/or out of the tube 1, preferably out of the needle 3.

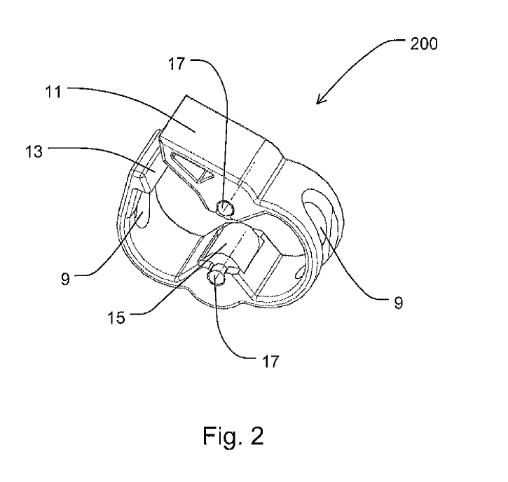

FIG. 2 shows a device 200 according to the present invention which in this embodiment example is made of or comprises an elastic material, e.g., an elastic plastic.

The device 200 is slid onto a tube 1 (see FIG. 1) in order to create a blood tube section 100 according to the present invention through use of openings 9 provided herein, which are merely exemplarily embodied each as openings having a closed circumference. During use of the blood tube section 100, the device 200 is moved on the tube 1 until the starting position for conveying the tube contents is reached. Then, a snap lock 11 is latched under a ledge 13, wherein latching is not necessary in other embodiments not illustrated here, or wherein latching may take place in one of several latching steps. In this position, the tube 1 which was passed through the openings 9 is squeezed between two rollers of which in FIG. 2 there is only one roller 15 illustrated. The rollers 15 are mounted in the roller bearings, here embodied as slide bearings, through the use of roller pins 17. If the device 200 is moved along the tube 1, the contents of the tube are conveyed by use of the device 200 in front of the rollers 15. The tube 1 is continuously squeezed between the rollers 15 along the path of movement, or its lumen is altered in its cross-section across the length of the tube or it is reduced in its cross-sectional area.

FIG. 3 shows schematically simplified a dialysis apparatus as an example for a treatment apparatus 300 according to the present invention.

During a dialysis treatment of a patient 500 by the treatment apparatus 300, two blood tube sections 100a (arterial) and 100v (venous) are connected with the treatment apparatus 300 through an extracorporeal blood circuit 25. The devices 200 are each in a position such that the corresponding tubes 1 (see FIG. 1) that are pushed through the openings 9 (see FIG. 2) are or will not be squeezed or altered in the cross-section of their lumina by the devices 200. The snap locks 11 (see FIG. 1) of the devices 200 are hereby each opened, i.e., not snapped into or locked under the ledge 13. Thus, blood from the device 200 may flow unhindered through the blood tube sections 100a and 100v.

The example of FIG. 3 shows two blood tube sections 100a and 100v according to the present invention. In fact, an extracorporeal blood circuit 25 may also comprise only one blood tube section according to the present invention instead of two, for instance, only the arterial blood tube section 100a or only the venous blood tube section 100v.

An exemplary procedure for returning blood that remains in the arterial blood tube section 100a at the end of the treatment is explained hereinafter. At the end of the dialysis treatment, the patient 500 is disconnected from the treatment apparatus 300 by disconnecting the arterial blood tube section 100a from the extracorporeal blood circuit 25. The blood pump 19 conveys the contents of the extracorporeal blood circuit 25 until a detector recognizes a limit between blood and air or possibly a saline solution or the like. Via the arterial blood tube section 100a which is still connected with the vascular system of the patient, a drug, e.g., EPO (erythropoetin), may be administered to the patient 500 manually. For this purpose, the device 200 of the arterial blood tube section 100a is moved as close as possible towards the corresponding shut-off device 23, the free end of the blood tube section 100a or a blood limit. Subsequently, the snap lock 11 is—depending on the design of the device 200—either latched into the ledge 13, or the device 200 is actuated in another way such that the tube 1 is squeezed by the rollers 15. Actuating in another way may occur such that the snap lock 11 is lowered such that the rollers 15 approach without the snap lock 11 being latched in or under the ledge 13 hereby. Finally, the device 200 is moved forward in the direction of the puncture of the patient 500 and the blood present therein is thereby supplied to the patient 500 together with the drug in the second direction of flow.

Optionally, this step of mostly draining the blood tube section 100 may be repeated several times, so that additional rest blood volume is supplied from the extracorporeal blood circuit 25 or the blood tube section 100 according to the present invention to the patient. Optionally, the circuit is vented at a suitable spot.

In the example of FIG. 3, the draining of the blood tube section 100 into the blood vessel system of the patient 500 is illustrated. However, also the draining of the blood tube section into receptacles, bags or containers is encompassed by the present invention, as far as the patient—different from the description here—is not to receive a drug via the blood tube section 100a.

Reference numeral list

| Reference | |
|---|---|
| numeral | Description |
| 100 | blood tube section |
| 100a | arterial blood tube section |
| 100v | venous blood tube section |
| 200 | conveying device |
| 300 | treatment apparatus |
| 500 | patient |
| 1 | tube |
| 3 | needle |
| 5a, 5b | connector, port |
| 7 | arrow which indicates moving directions |
| 9 | tube openings |
| 11 | snap lock |
| 13 | ledge |
| 15 | roller |
| 17 | roller pins |
| 19 | blood pump |
| 23 | shut-off device |
| 25 | extracorporeal blood circuit |

What is claimed is:

1. A blood tube section for extracorporeally carrying blood during a blood treatment of a patient, wherein the blood tube section comprises:
a tube with a lumen; and
at least one conveying device configured to convey blood, a drug solution or liquid mixtures within the blood tube section,
wherein said conveying device is configured to slide along at least a portion of the length of the tube and configured to deform or reduce a diameter or a cross-sectional area of the lumen of the tube.

2. The blood tube section according to claim 1, further comprising an element from the group consisting of:
one or more connectors for creating a fluid connection between the blood tube section and a needle for puncturing a blood vessel;
one or more connectors for creating a fluid connection between the blood tube section and a blood tube system;
one or more needles for creating a fluid connection between the blood tube section and a blood vessel of the patient; and
one or more puncturing aids for puncturing the blood vessel of the patient and/or for fixing the blood tube section to a body portion or skin portion of the patient.

3. The blood tube section according to claim 1, wherein the conveying device comprises a tubing clamp which is slidable along the portion of the length of the tube for the purpose of conveying blood.

4. The blood tube section according to claim 1, wherein the conveying device comprises one or more rollers for narrowing the lumen or for changing the cross-section of the tube.

5. The blood tube section according to claim 4, wherein the conveying device comprises two rollers.

6. The blood tube section according to claim 4, wherein at least one roller is configured to be detachable from said conveying device.

7. The blood tube section of claim 4, wherein each of said rollers is configured to be detachable from said conveying device.

8. The blood tube section according to claim 1, wherein the conveying device comprises an elastic material.

9. The blood tube section according to claim 1, further comprising an addition point for adding a liquid substance into the lumen of the tube.

10. An extracorporeal blood circuit, connected with at least one blood tube section according to claim 1.

11. A blood treatment apparatus for performing hemodialysis, hemofiltration or hemodiafiltration of a patient, wherein the blood treatment apparatus is connected with at least one blood tube section according to claim 1 and/or at least one extracorporeal blood circuit according to claim 10.

12. A method for conveying blood, a drug solution or a liquid mixture, comprising the steps of:
providing a blood tube section according to claim 1; and
manually sliding the conveying device along the portion of the length of the tube to convey said blood, drug solution or liquid mixture.

13. The method for conveying blood, a drug solution or a liquid mixture in accordance with claim 12, further comprising the steps of:
administering an active substance into the lumen of the tube; and
conveying the active substance within the lumen by said sliding of the conveying device.

14. A method for returning blood present in a blood tube, a blood tube system or an extracorporeal blood circuit to a patient, comprising the steps of:
providing a blood tube section according to claim 1 which is in fluid connection with a blood vessel of the patient; and
manually sliding the device for conveying blood present in the blood tube section along the portion of the length of the tube to convey blood present in the blood tube section into the vessel of the patient.

15. The method according to claim 14, wherein the conveying of blood present in the blood tube section into the vessel of the patient occurs during or at the end of a blood treatment selected from the group consisting of hemodialysis, hemofiltration, and hemodiafiltration.

16. The method according to claim 15, wherein during said blood treatment, the blood flows in a first direction of flow through the blood tube section, and the manual sliding of the conveying device causes blood to flow in a second direction of flow which is inverse to the first direction of flow.

17. The method according claim 14, wherein the conveying device is manually slided after disconnecting or stopping a fluid connection between the blood tube section and an extracorporeal blood circuit.
